(19) United States
(12) Patent Application Publication
(10) Pub. No.: US 2017/0325661 A1
(43) Pub. Date: Nov. 16, 2017

(54) ENDOSCOPIC METHOD AND DEVICE

(71) Applicant: MEDJET LTD., Yehud (IL)

(72) Inventors: Alexander POLYAKOV, Qidmat Tsvi (IL); Izhak YANIV, Ganei Tikva (IL)

(21) Appl. No.: 15/613,225

(22) Filed: Jun. 4, 2017

Related U.S. Application Data
(63) Continuation of application No. 13/823,511, filed on Mar. 14, 2013, now abandoned.
(60) Provisional application No. 61/405,760, filed on Oct. 22, 2010.

(51) Int. Cl. A61B 1/00; A61B 1/015; A61B 17/3203; A61C 17/02; A61B 1/273; A61B 1/31; A61B 1/05; A61B 1/005; A61B 17/00; A61B 17/12; A61B 17/22

(52) U.S. Cl. CPC A61B 1/00064; A61C 17/02; A61B 1/015; A61B 1/00091; A61B 17/3203; A61B 1/2736; A61B 1/2733; A61B 1/05; A61B 1/0051; A61B 2017/0034; A61B 2017/12004; A61B 2017/22037; A61B 2017/32035; A61B 1/31

(57) ABSTRACT

Disclosed are methods for performing endoscopy comprising directing a distal end of an endoscope to proximity of a portion of a surface of a bodily cavity; and directing a liquid-gas stream at a portion of the surface of the bodily cavity. Disclosed are also devices useful in endoscopy, comprising: an elongated body having a proximal end and a distal end; a nozzle at the distal end; a gas channel configured for conveying a gas from the proximal end to the nozzle; and a liquid channel configured for conveying a liquid from the proximal end to the nozzle, wherein the nozzle is configured to generate a liquid-gas stream directed in a specified direction from a gas conveyed by the gas channel and a liquid conveyed by the liquid channel.

### RELATED APPLICATION

The present application gains priority from U.S. Provisional Patent Application No. 61/405,760 filed 22 Oct. 2010, which is included by reference as if fully set forth herein.

### FIELD AND BACKGROUND OF THE INVENTION

The invention, in some embodiments, relates to the field of endoscopy and endoscopes. More particularly, but not exclusively, some embodiments of the invention relate to methods and devices that ease visual inspection of a bodily cavity, especially a cavity that is a lumen of the gastrointestinal tract, by moving an interfering object, such as fluid, semi-solid or solid interfering objects.

Endoscopes are instruments used for accessing the interior of bodily cavities such as the inside of a hollow organ, for example to inspect the lumen and luminal surfaces of parts of the gastrointestinal tract, respiratory tract, urinary tract, reproductive system or inside the abdomen. Endoscopcs are usually named according to the organ which these are used to inspect, e.g., colonoscope, sigmoidoscope, enteroscope, esophagogastroduodenoscopy, bronchoscope, falloposcope and laparoscope.

Typically, an endoscope is an elongated and flexible device having a maneuverable distal tip that allows the endoscope to be progressively pushed into and maneuvered inside the body of a subject. At the distal tip of the endoscope there is typically an illuminator and an imager, allowing an operator to visually inspect the area near the distal tip of the endoscope.

A problem encountered during endoscopy, especially of the gastrointestinal tract, is the presence of obscuring objects inside the bodily cavity, for example fluid, semi-solid or solid materials such as faeces and intestinal exudates that prevent rigorous inspection of the cavity surfaces, for example for the identification of polyps, bleeding or cancers.

The importance of maximizing the detection of abnormalities during colonoscopy cannot be understated: colon cancer is the second most common cancer in industrialized countries. It is important to prevent obscuring of the colon surface to improve the detection of abnormalities such as polyps, tumors and neoplastic lesions.

It is known to use colon cleansing preparations, for example, osmotic laxatives, polyethylene glycol (PEG) and stimulant laxatives. None of these preparations guarantee proper bowel preparation. Studies evaluating the effect of colonoscopy preparation quality on the detection of suspected colonic neoplasia show that polyp detection is dependent on the quality of bowel preparation and that more than 30% of incomplete colonoscopies are due to insufficient bowel preparation.

It is known to wash an inspected area, for example during colonoscopy, with an excess of clean liquid such as saline. To this end, colonoscopes and the like typically comprise a washing liquid channel through which a washing liquid such as saline can be directed to clean a bodily lumen. The large volume of water that must be used before and during the colonoscopy makes the procedure unpleasant and is not always effective.

To handle the large amount of liquid released and to clear away the obscuring objects, some endoscopes include a suction channel to remove the liquid. Such a suction channel is difficult to implement in the modest dimensions of an endoscope and may become blocked and ineffective.

It has been found that known methods of endoscopic inspection such as described above are insufficient: in randomized controlled clinical trials, inadequate colon cleansing was found to occur in between 10% and 75% of the cases (Froehlich F et al. in Gastrointestinal Endoscopy 2005, 61(3)).

### SUMMARY OF THE INVENTION

Some embodiments of the invention relate to methods and devices useful in endoscopy that, in some aspects, have advantages over known methods and devices. Some embodiments of the invention relate to methods and devices that ease visual inspection of a bodily cavity, especially a cavity that is a lumen of the gastrointestinal tract, by moving an interfering object, such as fluid, semi-solid or solid interfering objects.

Thus, according to an aspect of some embodiments of the invention, there is provided a method for performing endoscopy, comprising:
a) directing a distal end of an endoscope to proximity of a portion of a surface of a bodily cavity (in some embodiments of a human, in some embodiments of a non-human animal); and
b) directing a liquid-gas stream at the portion of the surface of the bodily cavity.
   In some embodiments, the method further comprises at least one of:
c) when the portion of the surface is obscured by the presence of an object, moving the object by the directing of the liquid-gas stream at the object, so the portion of a surface is less obscured;
d) when the portion of the surface is bleeding, cooling the portion by the directing of the liquid-gas stream so as to reduce the rate of the bleeding;
e) providing the liquid-gas stream wherein the liquid-gas stream comprises a stain suitable for use in chromoendoscopy, and marking the portion of the surface with the stain by the directing of the liquid-gas stream; and/or
f) providing the liquid-gas stream wherein the liquid-gas stream comprises an active pharmaceutical ingredient, and administering the active pharmaceutical ingredient to the portion of the surface by the directing of the liquid-gas stream.

The bodily cavity is any endoscopically-accessible bodily cavity. In some embodiments, the bodily cavity is a lumen of a gastrointestinal tract.

According to an aspect of some embodiments of the invention, there is also provided a device useful in endoscopy, comprising:
a) an elongated body having a proximal end and a distal end configured for maneuvering the distal end into a bodily cavity;
b) a nozzle at the distal end of the elongated body;
c) a gas channel configured for conveying a gas from the proximal end of the elongated body to the nozzle; and
d) a liquid channel configured for conveying a liquid from the proximal end of the elongated body to the nozzle;
   wherein the nozzle is configured to generate a liquid-gas stream directed in a specified direction from a gas conveyed by the gas channel and a liquid conveyed by the liquid channel, the directed liquid-gas stream sufficient for moving a resting object from a surface of a bodily cavity, without substantially damaging the surface.

Aspects and embodiments of the invention are described in the specification hereinbelow and in the appended claims.

Unless otherwise defined, all technical and scientific terms used herein have the same meaning as commonly understood by one of ordinary skill in the art to which the invention pertains. In case of conflict, the specification, including definitions, takes precedence.

As used herein, in some instances (for example in the priority document U.S. Provisional Patent Application 61/405,760 filed 22 Oct. 2010) the terms “bearing” (e.g., of a liquid or gas) and variants thereof are substantially synonymous with the term “conveying” and variants thereof.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

### BRIEF DESCRIPTION OF THE FIGURES

Some embodiments of the invention are herein described, by way of example only, with reference to the accompanying figures. The description, together with the figures, makes apparent how embodiments of the invention may be practiced to a person having ordinary skill in the art. The figures are for the purpose of illustrative discussion of embodiments of the invention and no attempt is made to show structural details of an embodiment in more detail than is necessary for a fundamental understanding of the invention. For the sake of clarity, some objects depicted in the figures are not to scale.

In the Figures:

FIG. 1A is a schematic depiction of an embodiment of a device as described herein for use together with an endoscope;

FIGS. 1B-1H are depictions of a nozzle assembly of a device of FIG. 1A;

FIG. 2A is a schematic depiction of a colonoscope for use together with a device of FIGS. 1A-1H;

FIG. 2B is a schematic depiction of a device of FIGS. 1A-1H in use together with a colonoscope of FIG. 2A;

FIG. 3A is a schematic depiction of an embodiment of a device as described herein that constitutes an endoscope; and

FIG. 3B is a schematic depiction of a nozzle assembly of a device of FIG. 3A in side cross section.

### DESCRIPTION OF SOME EMBODIMENTS OF THE INVENTION

Some embodiments of the invention relate to methods and devices useful in endoscopy that, in some aspects, have advantages over known methods and devices. Some embodiments of the invention relate to methods and devices that ease visual inspection of a bodily cavity, especially a cavity that is a lumen of the gastrointestinal tract, by moving an obscuring object, such as fluid, semi-solid or solid interfering objects.

The principles, uses and implementations of the teachings herein may be better understood with reference to the accompanying description and figures. Upon perusal of the description herein, one skilled in the art is able to implement the invention without undue effort or experimentation. In the figures, like reference numerals refer to like parts throughout.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

### Method for Performing Endoscopy

According to an aspect of some embodiments of the invention, there is provided a method for performing endoscopy, comprising:

a) directing a distal end of an endoscope to proximity of a portion of a surface of a bodily cavity (in some embodiments of a human, in some embodiments of a non-human animal); and

b) directing a liquid-gas stream at the portion of the surface of the bodily cavity.

In some embodiments, the method further comprises at least one of:

c) when the portion of the surface is obscured by the presence of an object, moving the object by the directing of the liquid-gas stream at the object, so the portion of a surface is less obscured;

d) when the portion of the surface is bleeding, cooling the portion by the directing of the liquid-gas stream so as to reduce the rate of the bleeding;

e) providing the liquid-gas stream wherein the liquid-gas stream comprises a stain suitable for use in chromoendoscopy, and marking the portion of the surface with the stain by the directing of the liquid-gas stream; and/or

f) providing the liquid-gas stream wherein the liquid-gas stream comprises an active pharmaceutical ingredient, and administering the active pharmaceutical ingredient to the portion of the surface by the directing of the liquid-gas stream.

The bodily cavity is any endoscopically-accessible bodily cavity. In some embodiments, the bodily cavity is a lumen of a gastrointestinal tract, for example a lumen of of the rectum, colon, large intestine or duodenum. As will be understood from the below, the teachings herein are exceptionally useful when implemented in bodily cavity that is a lumen of a gastrointestinal tract, the methods as described herein are exceptionally useful.

### Moving Obscuring Objects

Liquid-gas streams known in the art (see for example U.S. Pat. No. 6,283,936; U.S. Pat. No. 6,673,081; PCT Patent Application PCT/IL2005/000017; PCT Patent Application PCT/IL2006/000557 and US patent publication 2009/0234269) are typically used to damage tough and resilient tissue such as skin, for example to abrade such tissue. Surprisingly, it has been found that application of directed liquid-gas streams in accordance with the teachings herein during endoscopy such as colonoscopy effectively move obstructing objects like faeces and intestinal exudates, including disintegration of faeces, allowing inspection of the cavity surfaces yet does not substantially damage the thin and sensitive intestinal mucosa.

As discussed in the introduction, know endoscopic inspection of the surfaces of the gastrointestinal tract is difficult due to the presence of large amounts of interfering objects, typically fluid, semi-solid or solid materials such as faeces and intestinal exudates that prevent rigorous inspection of the cavity surfaces. In the art, it is known to clear the gastrointestinal tract of obstructing objects by irrigating with large volumes of washing liquids (e.g., using an enema, or ClearPath™ devices by Easy-Glide Ltd. (Bonita Springs, Fla., USA) or Jetprep flushing device by JetPrep Ltd., Herzliya, Israel).

In contrast, the teachings herein typically use only a small volume of liquid (saving time, reducing discomfort and mess, avoiding obscuring of view by the washing liquid) not to clear the gastrointestinal tract but to effectively disintegrate and move objects such as faeces that obscure the view of a portion of a gastrointestinal surfaces to be inspected. Additionally, the teachings herein also allow the effective inspection of features such as folds, creases and crevices by clearing obstructions therefrom: it is known that prior methods are ineffective in clearing objects from such features.

Thus, as noted above, in some embodiments, the method is implemented so that the liquid-gas stream moves an object that is obscuring the portion of the surface so the portion of a surface is less obscured, allowing inspection (e.g., endoscopic inspection) of the portion of the surface. Typically, such objects are resting (as opposed to being attached) on the surface and include liquid, semi-liquid and solid objects. When the bodily cavity is a gastrointestinal lumen, typical such objects are faeces and intestinal exudates that prevent rigorous inspection of the cavity, surfaces faeces, and intestinal exudates that prevent rigorous inspection of the surfaces of the bodily cavity. In some such embodiments, subsequent to the moving, the object is removed, for example using suction, for example through the working channel of an endoscope.

### Reducing Bleeding

In the art of endoscopy, such as colonoscopy, it is known that it may be difficult to inspect a portion of the surface of a bodily cavity due to bleeding of an ulcerous wound. In the art of endoscopy, such as colonoscopy, it is known to perform a surgical intervention, such as excision of tissue (e.g., removal of a polyp) from a portion of the surface of a bodily cavity that may be followed by bleeding of the portion.

It has been found that in some embodiments the liquid-gas stream can cool a portion of tissue by between about 5° C. and 15° C., usually between about 7° C. and about 10° C. It has been found that, in some embodiments, such cooling is sufficient to cause substantial contraction of blood vessels such as intestinal mucosa blood vessels to the extent that bleeding of a wound such as an ulcerous wound is reduced or stopped sufficiently long for inspection of the wound. It has also been found that, in some embodiments, such cooling is sufficient to cause substantial contraction of blood vessels to the extent that bleeding from a wound caused by excision of tissue is reduced or stopped. Surprisingly, in some embodiments such cooling has been found to be as effective as the topical application of adrenaline usually performed in the art.

Thus, as noted above, in some embodiments of the method described herein, the portion of the surface of the bodily cavity is bleeding and the method is implemented so that the liquid-gas stream cools the portion (i.e., selectively cools only the portion) so as to reduce the rate of (or even entirely stop) the bleeding. In some embodiments, the method further comprises prior to the directing of the liquid-gas stream, excising tissue (e.g., polyp, lesion, cancerous lesions) from the portion of the surface, thereby causing the portion of the surface to bleed.

### Chromoendoscopy

A known variant of endoscopy is chromoendoscopy, the application of a stain to the surface of a bodily cavity, e.g., for diagnosis. In gastrointestinal endoscopy (e.g., colonoscopy) a gastrointestinal stain is applied to a surface of the lumen of a portion of the gastrointestinal tract.

Some such stains (e.g., indigo carmine) are used as contrasting agents, allowing easier inspection of gastrointestinal surfaces, mainly in the stomach, colon and small intestine, useful for screening of early cancer, differentiation between malignant and benign lesions, pit-pattern analysis, diagnosis of spread and depth, observation of inflammatory diseases and evaluation of ulcer healing.

Some stains have different affinity to different types of tissue, e.g., healthy vs. pathological (cancerous tissue) and allow identification of pathological tissue with a greater degree of certainty, often obviating the need for biopsy. For example: Lugol's solution is used to stain normal squamous cells with glycogen, allowing for screening of early esophageal cancer, reflux esophagitis and columnar epithelium in the esophagus; methylene blue is used to stain small- and large-intestine cells and intestinal metaplasia for screening of intestinal metaplasia and ectopic gastric mucosa in the duodenum; and Toluidine blue is used to stain cellular nuclei for screening of squamous-cell carcinoma, reflux esophagitis, gastric or intestinal metaplasia in Barrett's esophagus.

It has been found that the teachings herein can be advantageously used for marking a portion of a surface of a bodily lumen by adding a stain suitable for chromoendoscopy to the liquid-gas stream. Some embodiments allow selective marking of only a desired portion of the surface. In some embodiments, the small amount of liquid allows neat and non-messy marking. Although not wishing to be held to any one theory, it is currently believed that in some embodiments such marking of a portion of a surface with a stain is exceptionally effective due to the velocity with which liquid particles in the liquid-gas stream impact the surface and/or due to the size of the liquid particles in the liquid-gas stream.

Thus, as noted above, in some embodiments the liquid-gas stream comprises a stain suitable for use in chromoendoscopy, and the directing of the liquid-gas stream leads to marking the portion (selectively marking only the portion) of the surface with the stain. In some embodiments, the bodily cavity is a lumen of the gastrointestinal tract and the stain is a gastrointestinal stain.

### Administration of Active Pharmaceutical Ingredients

In a manner analogous to marking of a portion of a surface of a bodily cavity with a stain, the teachings herein may be applied to the topical administration of an active pharmaceutical ingredient (API) to a portion of a surface of a bodily cavity, for example an anti-inflammatory or anti-cancer API. Although not wishing to be held to any one theory, it is currently believed that in some embodiments such administration of an API is exceptionally effective due to the velocity with which liquid particles in the liquid-gas stream impact the surface and/or due to the size of the liquid particles in the liquid-gas stream.

Thus, as noted above, in some embodiments the liquid-gas stream comprises an API, and the directing of the liquid-gas stream leads to administration of the active pharmaceutical ingredient to the portion of the surface, typically, selective administration to only the portion.

Liquid-Gas Stream Any suitable liquid-gas stream may be used in implementing the teachings herein.

Typically, the liquid-gas stream is a two-phase stream of particles of a liquid component of the liquid-gas stream in a gas component of the liquid-gas stream.

In some embodiments, the liquid-gas stream comprises a mist having a velocity in a specified direction. In some embodiments, the mist comprises particles of liquid having a mean particle diameter of less than about 20 micrometers, less than about 10 micrometers, less than about 8 micrometers, less than about 5 micrometers, less than about 3 micrometers and even less than about 1 micrometers. In some embodiments, the mist comprises particles of liquid having a mean particle diameter of between about 5 micrometers and about 20 micrometers.

The velocity of the liquid-gas stream is any suitable velocity in a specified direction. In some embodiments, velocity of the liquid-gas stream is at least about 100 m/sec, at least about 150 m/sec and even at least about 175 m/sec. In some embodiments, the velocity of the liquid-gas stream is not more than about 300 m/sec, not more than about 250 m/sec and even not more than about 225 m/sec.

The size of the portion of the surface of the bodily cavity that is affected by the liquid-gas stream at any moment (i.e., determined by the dimensions of the liquid-gas stream) is typically small, usually not greater than about 80 mm² (equivalent to a circle having a 5 mm radius), not greater than about 50 mm² (equivalent to a circle having a 4 mm radius) and even not greater than about 20 mm² (equivalent to a circle having a 2.5 mm radius).

### Gas Component of Liquid-Gas Stream

Any suitable gas flow rate may be used in implementing the liquid-gas stream of the teachings herein.

In some embodiments (e.g., in some embodiments where the bodily cavity is a lumen of the gastrointestinal tract) the flow rate of the gas in the liquid-gas stream is at least about 0.5, at least about 0.6 and even at least about 0.7 l/minute.

In some embodiments (e.g., in some embodiments where the bodily cavity is a lumen of the gastrointestinal tract) the flow rate of the liquid in the liquid-gas stream is not more than about 2, not more than about 1.8 and even not more than about 1.5 l/minute.

In some embodiments (e.g., in some embodiments where the bodily cavity is a lumen of the gastrointestinal tract) the flow rate of the liquid in the liquid-gas stream is between about 0.5 and about 2 l/minute, between about 0.6 and about 1.8 μminute and even between about 0.7 and about 1.5 l/minute.

Any suitable gas may be used in implementing the teachings herein as a gas component of the liquid-gas stream, in some embodiments comprising at least one gas selected from the group consisting of carbon dioxide, air, nitrogen, helium, neon, argon, krypton, xenon and mixtures thereof.

As detailed hereinbelow, an exceptional advantage of some embodiments is that the gas component of the gas-liquid stream helps maintain or leads to insufflation of the bodily cavity, especially when the gas component is carbon dioxide. In the art, carbon dioxide is typically used as an insufflation gas, especially in the field of gastrointestinal endoscopy. It has been found that carbon dioxide is readily absorbed by intestinal mucosal tissue into the blood stream and cleared by the respiratory system. Accordingly, when carbon dioxide is used as an insufflation gas there is less patient bloating, less pain and quicker patient recovery. Thus, in some embodiments, a gas component of the liquid-gas stream comprises carbon dioxide and in some embodiments, a gas component of the liquid-gas stream is substantially pure (in some embodiments medical-grade) carbon dioxide that is typically used as an insufflation gas.

### Liquid Component of Liquid-Gas Stream

Any suitable liquid flow rate may be used in implementing the liquid-gas stream of the teachings herein. In some embodiments (e.g., in some embodiments where the bodily cavity is a lumen of the gastrointestinal tract) the flow rate of the liquid in the liquid-gas stream is at least about 0.1, at least about 0.2, at least about 0.3, at least about 0.4 and even at least about 0.5 ml/minute. In some embodiments (e.g., in some embodiments where the bodily cavity is a lumen of the gastrointestinal tract) the flow rate of the liquid in the liquid-gas stream is not more than about 2, not more than about 1.8, not more than about 1.5, not more than about 1.3 and even not more than about 1.2 ml/minute. In some embodiments (e.g., in some embodiments where the bodily cavity is a lumen of the gastrointestinal tract) the flow rate of the liquid in the liquid-gas stream is between about 0.1 and about 2 ml/minute, between about 0.2 and about 1.8 ml/minute, between about 0.3 and about 1.5 ml/minute, between about 0.4 and about 1.3 ml/minute and even between about 0.5 and about 1.2 ml/minute.

Any suitable liquid may be used in implementing the teachings herein as a liquid component of the liquid-gas stream. In some embodiments, the liquid component of the liquid-gas stream comprises at least one liquid selected from the group consisting of water, saline, PBS, a gastrointestinal stain suitable for use in chromoendoscopy and a pharmaceutical composition including an active pharmaceutical ingredient for administration in the gastrointestinal tract.

In some embodiments, the liquid is isotonic with animal tissue. That said, in some embodiments the liquid is hypertonic or hypotonic with animal tissue.

In some embodiments, the liquid is sterile. That said, in some embodiments the liquid is non-sterile, for example when the bodily cavity is the gastrointestinal tract lumen.

In some embodiments, the liquid is saline as being typically readily available in health-care settings, is isotonic with animal tissue and is sterile.

In some embodiments, the liquid is water, including non-sterile water, as being typically readily available.

In some embodiments, the liquid is demineralized (e.g., triple-distilled) water in order to reduce the chance of clogging of the component (e.g., a nozzle) from which the gas-liquid stream emerges.

### Insufflation

As known in the art, endoscopy, especially a gastrointestinal endoscopy, is typically performed during insufflation that is to say, a continuous or intermittent stream of insufflation gas is directed into the bodily cavity to “inflate” the bodily cavity. Although any gas may be used as an insufflation gas, carbon dioxide is the preferred insufflation gas due to quick absorption by the body as described above.

In some embodiments, the method further comprises releasing an insufflation gas inside the bodily cavity to maintain insufflation thereof. In some embodiments, the insufflation gas is released inside the bodily cavity in the usual way as is known in the art.

An inherent advantage of some embodiments of the teachings herein is that the gas component of the liquid-gas stream may be used to insufflate or maintain insufflation of a bodily cavity.

In some embodiments, the liquid-gas stream is generated intermittently when needed (e.g., to move an obstruction) and the gas component thereby released in the bodily cavity functions as a (supplementary) insufflation gas.

In some embodiments, the releasing of an insufflation gas includes continuously generating the liquid-gas stream inside the bodily cavity, so that the gas component thereby released in the bodily cavity functions as a (supplementary) insufflation gas.

In some embodiments, the releasing of an insufflation gas includes continuously generating a stream of gas inside the bodily cavity and intermittently adding liquid to the stream of gas to generate the liquid-gas stream, so that the gas component thereby released in the bodily cavity functions as a (supplementary) insufflation gas.

The method described herein may be implemented using any suitable device. In some embodiments, the method is preferably implemented using a device as described herein.

### Device Useful in Endoscopy

According to an aspect of some embodiments of the invention, there is also provided a device useful in endoscopy, comprising:

a) an elongated body having a proximal end and a distal end configured for maneuvering the distal end into a bodily cavity;

b) a nozzle at the distal end of the elongated body;

c) a gas channel configured for conveying a gas from the proximal end of the elongated body to the nozzle; and

d) a liquid channel configured for conveying a liquid from the proximal end of the elongated body to the nozzle;

wherein the nozzle is configured to generate a liquid-gas stream directed in a specified direction from a gas conveyed by the gas channel and a liquid conveyed by the liquid channel, the directed liquid-gas stream sufficient for moving a resting object from a surface of a bodily cavity, without substantially damaging the surface. Objects are resting (as opposed to being attached) on the surface and include liquid, semi-liquid and solid objects. In some embodiments, the bodily cavity is a lumen of the gastrointestinal tract. When the bodily cavity is a gastrointestinal lumen, typical such objects are faeces and intestinal exudates.
Device that Constitutes an Endoscope

In some embodiments, the device substantially constitutes an endoscope, in some embodiments the elongated body is sufficiently flexible so that the device constitutes a flexible endoscope. In some such embodiments, the device substantially constitutes a gastrointestinal endoscope, in some embodiments selected from the group consisting of a colonoscope, a flexible sigmoidoscope, a gastrocope, and an enteroscope.

In some embodiments, an embodiment of a device that constitutes an endoscope is configured (analogously to known endoscopes) for inspecting a portion of a surface of a bodily cavity, further comprising an imager, and optionally an illuminator, at the distal end of the elongated body, typically but not necessarily at the distal tip of the elongated body.

The dimensions (length, outer diameter) of embodiments of a device that constitutes an endoscope are typically substantially the same as corresponding prior art endoscopes, for example a device as described herein that is substantially a colonoscope has a length and outer diameter that is equivalent to and determined by the same factors as a prior art colonoscope.

Device for Use with an Endoscope

In some embodiments, a device as described herein is a device configured for use together with an endoscope. Such embodiments allow a user to use any suitable endoscope (for example, a commercially-available endoscope, e.g., an endoscope that the user has available or prefers to use) to implement the teachings herein.

Thus, in some embodiments, the elongated body is configured (in terms of length, external diameter, flexibility, outer surface lubricity) for passage through a channel of an endoscope, distal end first, from a proximal end to a distal end thereof. In some embodiments, the elongated body is flexible and suitable for passage through a channel of a flexible endoscope. In some embodiments, the elongated body is configured for passage through a channel of a gastrointestinal endoscope, for example, a gastrointestinal endoscope selected from the group consisting of a colonoscope, a flexible sigmoidoscope, a gastrocope and an enteroscope.

Typically the device is configured for passage through the work channel found in a suitable endoscopes, although in some embodiments the device is configured for passage through another available endoscope channel.

As is clear to one skilled in the art, one important aspect of configuration for passage through an endoscope channel is that the outer diameter of the elongated body of the device be smaller than the inner diameter of the endo scope channel. Generally speaking, the elongated body of a device as described herein has an outer diameter of less than about 5 mm, less than about 4.5 mm, less than about 4 mm and even less than about 3.5 mm. For example, colonoscopes typically include a working channel having an inner diameter of 3.7 mm, so that the elongated body of a suitably-configured device has an outer diameter of less than 3.7 mm, for example 3.4 mm. For example, a gastroscope typically include a working channel having an inner diameter of 2.5 mm, so that the elongated body of a suitably-configured device has an outer diameter of less than 2.5 mm, for example 2.3 mm.

An additional aspect of configuration for passage through an endoscope channel is the length of the elongated body of the device. Specifically, the elongated body must be sufficiently long so that when the nozzle of the device is at or protrudes from the distal tip of an associated endoscope, at least a portion of the proximal end of the device protrudes from the proximal end of the endoscope. Preferably, the elongated body is not too long as this makes use thereof inconvenient. Generally speaking, the elongated body of a device as described herein is not less than about 80 cm, not less than about 85 cm and even not less than about 90 cm and not more than about 300 cm. For example, embodiments of devices configured for use with an enteroscope or a colonoscope are typically between about 150 cm and about 250 cm long while embodiments of devices configured for use with an sigmoidoscope or a gastroscope are typically between about 100 cm and about 150 cm long. In some such embodiments, the device is configured for reuse, e.g., for use on multiple occasions and/or for multiple patients, with or without cleaning between occasions and/or patients.

In some such embodiments, the device is configured for single-use, that is to say, is configured to be used once and then discarded. In typical such embodiments the device is provided singly-packaged in a sterility-preserving package as known in the art.

### Channels

The gas channel and the liquid channel are arranged in the elongated body in any suitable fashion.

In some embodiments, the gas channel and the liquid channel are substantially coaxial. For example, in some such embodiments a smaller diameter tube which bore constitutes the liquid channel passes through the bore of a larger diameter tube (in some embodiments which outer surface constitutes the outer surface of the elongated body) while the volume having a ring-shaped cross section defined between the inner surface of the larger-diameter tube and the outer surface of the smaller-diameter tube constitutes the gas channel.

In some embodiments, the gas channel and the liquid channel are substantially parallel. For example, in some such embodiments, the gas channel and the liquid channel are each defined by a different tube, for example the two tubes held inside the bore of a single larger tube that acts as a sheath, and/or physically associated, for example by welding, adhering or the like. For example, in some such embodiments, the gas channel and the liquid channel are each defined by a distinct channel passing through a single monolithic elongated member (e.g., made by extrusion).

The size of the gas channel and of the liquid channel, that is to say, the inner diameter of the bore that constitutes the respective channel is any suitable size. The sizes are typically determined with consideration of the maximal outer dimensions of the elongated body and the available materials from which to fashion the device.

In a typical embodiment (for example for a device configured to pass through the 3.7 mm working channel of a standard colonoscope), the cross sectional area of the liquid channel is not less than about 0.03 mm² (equivalent to a circle having a radius of about 0.2 mm), not less than about 0.07 mm² (equivalent to a circle having a radius of about 0.3 mm), and even not less than about 0.13 mm² (equivalent to a circle having a radius of about 0.4 mm). That said, in a typical embodiment (for example for a device configured to pass through the 3.7 mm working channel of a standard colonoscope), the cross sectional area of the liquid channel is not more than about 0.8 mm² (equivalent to a circle having a radius of about 1 mm), not more than about 0.5 mm² (equivalent to a circle having a radius of about 0.8 mm), and even not more than about 0.28 mm² (equivalent to a circle having a radius of about 0.6 mm).

In a typical embodiment (for example for a device configured to pass through the 3.7 mm working channel of a standard colonoscope), the cross sectional area of the gas channel is not less than about 0.2 mm² (equivalent to a circle having a radius of about 0.5 mm), not less than about 0.5 mm² (equivalent to a circle having a radius of about 0.8 mm), and even not less than about 0.8 mm² (equivalent to a circle having a radius of about 1 mm). That said, in a typical embodiment (for example for a device configured to pass through the 3.7 mm working channel of a standard colonoscope), the cross sectional area of the gas channel is not more than about 3.1 mm² (equivalent to a circle having a radius of about 2 mm), not more than about 2 mm² (equivalent to a circle having a radius of about 1.6 mm), and even not more than 1.5 mm² (equivalent to a circle having a radius of about 1.4 mm).

### Nozzle

As noted above, a nozzle is a component of a device useful in endoscopy as described herein that is positioned at the distal end (in some embodiments at a distal tip) of the elongated body. The nozzle is is configured to generate a directed liquid-gas stream from a gas conveyed by the gas channel and a liquid conveyed by the liquid channel, that is to say, directed in a specified direction. In some embodiments, the specified direction is a direction parallel to the axis of the distal end of the elongated body.

Typically, the nozzle is configured to generate a liquid-gas stream that is a two-phase stream of particles of the liquid conveyed to the nozzle through the liquid channel in the gas conveyed to the nozzle through the gas channel.

In some embodiments, the nozzle is configured to accelerate the gas received from the gas channel so that the gas is discharged from the nozzle at an elevated velocity. In some such embodiments, the nozzle is configured to accelerate the gas to near-sonic or to sonic velocities at the narrowest portion of the nozzle. In some such embodiments, the nozzle is also configured to discharge the liquid received from the liquid channel into the accelerated gas, leading to formation of liquid particles having the elevated velocity.

In some embodiments, the nozzle and the device are configured (in terms of nozzle architecture, channel diameters and the like) to generate a liquid-gas stream where the liquid-gas stream comprises a mist having a velocity in the specified direction. In some embodiments, the nozzle and the device are configured so that a generated mist comprises particles of liquid having a mean particle diameter of less than about 20 micrometers, less than about 10 micrometers, less than about 8 micrometers, less than about 5 micrometers, less than about 3 micrometers and even less than about 1 micrometers. In some embodiments, the nozzle and the device are configured to generate a liquid-gas stream a generated mist comprises particles of liquid having a mean particle diameter of between about 5 micrometers and about 20 micrometers.

In some embodiments, the nozzle and the device are configured to generate a liquid-gas stream having a velocity relative to the nozzle in the specified direction of at least about 100 m/sec, at least about 150 m/sec and even at least about 175 m/sec. In some embodiments, the nozzle and the device are configured to generate a liquid-gas stream having a velocity relative to the nozzle in the specified direction of not more than about 300 m/sec, not more than about 250 m/sec and even not more than about 225 m/sec.

In some embodiments, the nozzle is configured to generate a liquid-gas stream that is substantially divergent, that is to say, travels from the nozzle in the specified direction with a diameter that increases with increasing distance from the nozzle. In such embodiments, the angle of divergence is, typically not more than about 20° from parallel, not more than about 15° from parallel and even not more than about 12° from parallel with the specified direction. Typically, but not necessarily, the cross-section of the liquid-gas-stream perpendicular to the specified direction is circular.

The area of the cross-section of the liquid-gas stream perpendicular to the specified direction (a dimension that determines the size of a portion of a surface of a bodily cavity that is affected by the liquid-gas stream at any moment is typically small, when measured at a distance of 5 mm from the nozzle usually not greater than about 80 mm² (equivalent to a circle having a 5 mm radius), not greater than about 50 mm² (equivalent to a circle having a 4 mm radius) and even not greater than about 20 mm² (equivalent to a circle having a 2.5 mm radius).

Embodiments of a device as described herein are configured (especially in terms of architecture of the nozzle, gas channel and liquid channel) to generate a liquid-gas stream suitable for an intended use.

### Gas-Source

As noted above, in a device as described herein, a gas channel conveys a gas from the proximal end of the device to a nozzle at the distal end of the device to generate a gas-liquid stream. Typically, the conveyed gas is provided from a gas-source functionally associated with the gas channel at the proximal end of the distal body. In some embodiments, the gas-source is a component of the device. In some embodiments, the gas-source is not a component of the device. In some such embodiments, the device comprises a connector configured for reversible functional association of a gas-source with the gas channel at the proximal end of the elongated body.

The gas provided from a gas-source and from which the device is configured to generate the liquid-gas stream is any suitable gas. In some embodiments the gas comprises at least one gas selected from the group consisting of carbon dioxide, air, nitrogen, helium, neon, argon, krypton, xenon and mixtures thereof. As noted above, in some embodiments it is preferred that the gas comprise carbon dioxide and in some embodiments, substantially pure (in some embodiments medical-grade) carbon dioxide that is typically used as an insufflation gas.

In some embodiments (e.g., in some embodiments where the device is configured for gastrointestinal endoscopy), the device is configured so that a flow rate of gas in the generated liquid-gas stream from the nozzle is at least about 0.5, at least about 0.6 and even at least about 0.7 l/minute. In some embodiments (e.g., in some embodiments where the device is configured for gastrointestinal endoscopy), the device is configured so that a flow rate of gas in the generated liquid-gas stream from the nozzle is not more than about 2, not more than about 1.8 and even not more than about 1.5 l/minute. In some embodiments (e.g., in some embodiments where the device is configured for gastrointestinal endoscopy), the device is configured so that a flow rate of gas in the generated liquid-gas stream from the nozzle is between about 0.5 and about 2 l/minute, between about 0.6 and about 1.8 l/minute and even between about 0.7 and about 1.5 l/minute.

Typically, the device is configured so that the gas is conveyed at an elevated pressure to the nozzle. Any suitable gas pressure may be used. As noted above, in some embodiments, the device is configured so that the nozzle accelerates the gas received from the gas channel so that the gas is discharged from the nozzle at an elevated velocity, in some such embodiments, the nozzle is configured to accelerate the gas to near-sonic or to sonic velocities at the narrowest portion of the nozzle. In some such embodiments, the device is configured so that the gas is conveyed to the nozzle at a pressure of at least about 5 kPa (about 40 mm Hg) and even at least about 8 kPa (about 60 mm Hg). In some such embodiments, the device is configured so that the gas is conveyed to the nozzle at a pressure of not more than about 14 kPa (about 100 mm Hg) and even not more than about 11 kPa (about 80 mm Hg). In some such embodiments, the device is configured so that the gas is conveyed to the nozzle at a pressure of between about 5 kPa and about 14 kPa and in some embodiments at a pressure of between about 8 kPa and about 11 kPa.

For the gas to be conveyed at a suitable elevated pressure and at a suitable rate, in some embodiments the gas-source is pressurized, that is to say configured to drive the gas through the gas channel to the nozzle at a suitable pressure and rate, e.g., at the values listed above. In some embodiments, the gas-source comprises a pump. In some embodiments, the gas-source comprises a pressurized container, for example a gas bottle that typically contains the gas at an elevated pressure as a gas (e.g., in the case of air, argon, helium, krypton, xenon and nitrogen) or at least partially as a liquid (e.g., in the case of carbon dioxide)

### Liquid-Source

As noted above, in a device as described herein a liquid channel conveys a liquid from the proximal end of the device to a nozzle at the distal end of the device to generate a gas-liquid stream. Typically, the conveyed liquid is provided from a liquid-source functionally associated with the liquid channel at the proximal end of the distal body. In some embodiments, the liquid-source is a component of the device. In some embodiments, the liquid-source is not a component of the device. In some such embodiments, the device comprises a connector configured for reversible functional association of a liquid-source with the liquid channel at the proximal end of the elongated body.

The liquid provided from a liquid-source and from which the device is configured to generate the liquid-gas stream is any suitable liquid. In some embodiments the liquid comprises at least one liquid selected from the group consisting of water, saline, PBS, a gastrointestinal stain suitable for use in chromoendoscopy and a pharmaceutical composition including an active pharmaceutical ingredient for administration in the gastrointestinal tract.

In some embodiments (e.g., in some embodiments where the device is configured for gastrointestinal endoscopy), the device is configured so that a flow rate of liquid in the generated liquid-gas stream from the nozzle is at least about 0.1, at least about 0.2, at least about 0.3, at least about 0.4 and even at least about 0.5 ml/minute. In some embodiments (e.g., in some embodiments where the device is configured for gastrointestinal endoscopy), the device is configured so that a flow rate of liquid in the generated liquid-gas stream from the nozzle is not more than about 2, not more than about 1.8, not more than about 1.5, not more than about 1.3 and even not more than about 1.2 ml/minute. In some embodiments (e.g., in some embodiments where the device is configured for gastrointestinal endoscopy), the device is configured so that a flow rate of liquid in the generated liquid-gas stream from the nozzle is between about 0.1 and about 2 ml/minute, between about 0.2 and about 1.8 ml/minute, between about 0.3 and about 1.5 ml/minute, between about 0.4 and about 1.3 ml/minute and even between about 0.5 and about 1.2 ml/minute.

### Non Pressurized Liquid-Source

In some embodiments, the device is configured for use with a non-pressurized liquid-source for providing a liquid to be conveyed by the liquid channel and the corresponding liquid-source is not pressurized, that is to say, absent an additional force, the liquid passes through the liquid conduit and out of the nozzle only in insubstantial amounts, for example by evaporation or capillary action. In some such embodiments, the liquid-source is a collapsible container (e.g., a bag of saline) maintained at the same height (within 10 mm) or lower than the nozzle.

In some such embodiments, the device is configured to draw the liquid from the non-pressurized liquid-source, drive the drawn through the liquid channel and to the nozzle to generate the liquid-gas stream. For example, in some such embodiments, a device is configured for use with a pressurized gas-source, and the nozzle is configured so that passage of the gas from the gas channel through the nozzle draws the liquid from the liquid channel through the nozzle, to generate the liquid-gas stream. In some such embodiments, the liquid is drawn from the liquid channel by a Venturi effect, in a manner similar to an atomizer nozzle, for example as known in the art of airbrushing or carburetors. Some such embodiments are preferred as these are simple to implement and operate, and have been found to generate a liquid-gas stream suitable for the endoscopic uses as described herein.

### Pressurized Liquid-Source

In some embodiments, the device is configured for use with a pressurized liquid-source for providing a liquid to be conveyed by the liquid channel and the corresponding liquid-source is pressurized, that is to say, when activated (e.g., a switch functionally associated with the liquid-source is turned on, a valve is opened), the liquid is driven through the liquid channel and out through the nozzle. In some such embodiments, the device is configured so that the pressure of the pressurized liquid-source drives a liquid through the liquid channel to the nozzle to generate the liquid-gas stream. An advantage of some such embodiments is that the rate of liquid conveyed to the nozzle is meterable and/or controllable, especially useful for dispensing a specific amount of liquid such as a liquid that comprises a stain or an active pharmaceutical ingredient. In some such embodiments, the pressurized liquid-source comprises a pump (e.g., a peristaltic pump, a syringe pump, a component that “squeezes” a collapsible container such as a bag of saline in a meterable way). In some such embodiments, the pressurized liquid-source comprises a liquid in a closed container under a chamber holding a pressurized gas. In some such embodiments, the pressurized liquid-source comprises a collapsible container such as a bag of saline maintained higher (at least 10 mm) than the nozzle so that the force applied by gravity to the liquid in the container “pressurizes” the liquid source. In some embodiments, the pressurized liquid-source comprises a collapsible container (such as a bag of saline) compressed substantially indiscriminately (e.g., by inflating a blood-pressure measuring cuff that encircles the collapsible container) to apply a force to the liquid in the container that “pressurizes” the liquid source.

In some such embodiments (e.g., in some embodiments where the device is configured for gastrointestinal endoscopy), the pressurized liquid-source is configured to drive a liquid through the liquid channel and to the nozzle at a rate of at least about 0.1, at least about 0.2, at least about 0.3, at least about 0.4 and even at least about 0.5 ml/minute. In some embodiments (e.g., in some embodiments where the device is configured for gastrointestinal endoscopy), the pressurized liquid-source is configured to drive a liquid through the liquid channel and to the nozzle at a rate of not more than about 2, not more than about 1.8, not more than about 1.5, not more than about 1.3 and even not more than about 1.2 ml/minute. In some embodiments (e.g., in some embodiments where the device is configured for gastrointestinal endoscopy), the pressurized liquid-source is configured to drive a liquid through the liquid channel and to the nozzle at a rate of between about 0.1 and about 2 ml/minute, between about 0.2 and about 1.8 ml/minute, between about 0.3 and about 1.5 ml/minute, between about 0.4 and about 1.3 ml/minute and even between about 0.5 and about 1.2 ml/minute.

### Activation Mechanism

In some embodiments, the device is configured that during use of the device a liquid-gas stream is continuously generated. In some such embodiments, the amount liquid in the liquid-gas stream is relatively modest and typically does not lead to substantial negative effects such as “flooding” of a bodily cavity, and the gas in the liquid-gas stream may assist in insufflation of a bodily cavity as discussed in detail hereinbelow. For use, the nozzle of such a device is typically introduced into a bodily cavity and then the device and associated components activated to allow conveying of gas and liquid to the nozzle through the respective channels to generate a liquid-gas stream. For example, an associated pressurized gas-source is activated to allow continuous conveying of gas from the gas-source to the nozzle and, if present, an associated pressurized liquid-source is activated to allow continuous conveying of liquid to the nozzle.

In some embodiments, the device is configured for on-demand generation of a liquid-gas stream, for example, comprises an activation mechanism (of any suitable technology such as an on/off switch or a valve such as solenoid valve, ball valve, gate valve) for controlling generation of the liquid-gas stream, the activation mechanism having at least two states, an off state during which a liquid-gas stream is not generated and an on state during which a liquid-gas stream is generated. In some such embodiments, the activation mechanism is biased to the off state and requires an action to set and maintain in the on state, e.g., by using a trigger such as a foot pedal or a switch. For use, the nozzle of such a device is typically introduced into a bodily cavity and then the device and associated components activated with the activation mechanism biased to the off state so that no liquid-gas stream is generated. The operator performs endoscopy in the usual way. When the operator decides to implement the teachings herein, for example, observes and obscuring object resting on a surface of the bodily cavity, the operator sets the activation mechanism to the on state e.g. by using a trigger, generating a liquid-gas stream to move the obscuring object. Once the obscuring object is moved, the operator sets the activation mechanism to the off state (in some embodiments, simply by releasing the trigger) so that a liquid-gas stream is no longer generated.

### Activation Mechanism for Preventing Liquid Conveying

In some embodiments, an activation mechanism is configured to prevent the conveying of liquid from the liquid-source to the nozzle when in the off state.

In some such embodiments, the liquid-source is a pressurized liquid-source and the activation mechanism comprises a switch having at least two states: an off state that deactivates the pressurized liquid-source (e.g., sets a pump to an off state) when the activation mechanism is in an off state preventing the conveying of liquid from the liquid-source to the nozzle; and an on state that activates the pressurized liquid-source (e.g., sets a pump to an on state) when the activation mechanism is in an on state allowing the conveying of liquid from the liquid-source to the nozzle.

In some such embodiments, the activation mechanism comprises a liquid cut-off valve having at least two states: a closed state when the activation mechanism is in an off state that blocks the conveying of liquid from the liquid-source to the nozzle; and an open state when the activation mechanism is in an on state that allows the conveying of liquid from the liquid-source to the nozzle. In some such embodiments, such a liquid cut-off valve is located between the liquid-source and the proximal end of the elongated body of the device.

In some such embodiments, the device is configured that when the activation mechanism is in the off state and preventing the conveying of liquid from the liquid-source to the nozzle, conveying of gas from the gas-source to the nozzle is (in some embodiments, optionally) not interrupted. In such embodiments, gas is continuously conveyed into the bodily cavity, for example, possibly assisting in insufflation of a bodily cavity as discussed in detail hereinbelow.

### Activation Mechanism for Preventing Gas Conveying

In some embodiments, an activation mechanism is configured to prevent the conveying of gas from the gas-source to the nozzle when in the off state.

In some such embodiments, the gas-source is a pressurized gas-source and the activation mechanism comprises a switch having at least two states: an off state that deactivates the pressurized gas-source (e.g., sets a pump to an off state) when the activation mechanism is in an off state preventing the conveying of gas from the gas-source to the nozzle; and an on state that activates the pressurized gas-source (e.g., sets a pump to an on state) when the activation mechanism is in an on state allowing the conveying of gas from the gas-source to the nozzle.

In some such embodiments, the activation mechanism comprises a gas cut-off valve having at least two states: a closed state when the activation mechanism is in an off state that blocks the conveying of gas from the gas-source to the nozzle; and an open state when the activation mechanism is in an on state that allows the conveying of gas from the gas-source to the nozzle. In some such embodiments, such a gas cut-off valve is located between the gas-source and the proximal end of the elongated body of the device.

In some such embodiments, the device is configured that when the activation mechanism is in the off state and preventing the conveying of gas from the gas-source to the nozzle, conveying of liquid from the liquid-source to the nozzle is (in some embodiments, optionally) not interrupted. In some such embodiments, liquid is continuously conveyed into the bodily cavity.

### Activation Mechanism for Preventing Both Gas and Liquid Conveying

In some embodiments the activation mechanism is configured to prevent both the conveying of gas from the gas-source and the conveying of gas to the nozzle when in the off state. In some such embodiments, the activation mechanism is configured to concurrently prevent conveying of both the gas and the liquid. In some such embodiments, the activation mechanism is configured to optionally prevent conveying of gas but allow conveying of liquid, allowing conveying of liquid even when no liquid-gas stream is generated. In some such embodiments, the activation mechanism is configured to optionally prevent conveying of liquid but allow conveying of gas, allowing conveying of gas even when no liquid-gas stream is generated.

### Insufflation

As discussed above, endoscopy, especially a gastrointestinal endoscopy, is typically performed during insufflation of the bodily cavity. Although insufflation is sometimes implemented using a separate insufflation device, insufflation is typically implemented by providing an endoscope with a dedicated insufflation channel through which insufflation gas is conveyed from the proximal end to the distal end of the elongated body of the endoscope to be released inside the bodily cavity to effect insufflation.

In some embodiments, a device as described herein comprises an insufflation channel for conveying an insufflation gas from the proximal end to the distal end of the elongated body thereof.

In some embodiments where a device as described herein comprises an insufflation channel, the insufflation channel is a dedicated insufflation channel. During use, such an insufflation channel is functionally associated with an insufflation gas-source (e.g., an Endoflator electronic insufflation adapter by Karl Storz, Tuttlingen, Germany) and is used to effect insufflation of a bodily cavity substantially in the usual way. In some such embodiments, gas conveyed to the nozzle by the gas channel and released in the bodily cavity helps maintain insufflation of the bodily cavity.

In some embodiments where a device as described herein comprises an insufflation channel, the insufflation channel is the gas channel. In some such embodiments, the device is configured for substantially continuous conveying of gas from a gas-source through the gas channel and to the nozzle for release inside a bodily cavity. In some such embodiments, the device is configured for substantially continuous generation of a liquid-gas stream. That said, in some such embodiments, the device is configured for on-demand generation of a liquid-gas stream, that is to say, the device is configured to substantially continuously release a stream of gas in the bodily cavity to effect insufflation and configured to convey liquid to the nozzle to generate a liquid-gas stream only on-demand.

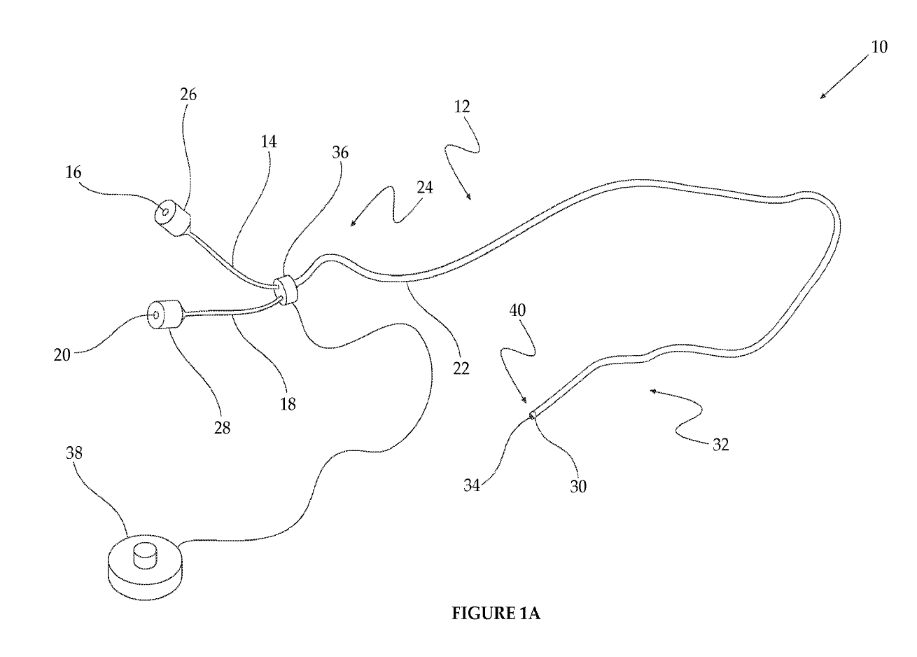

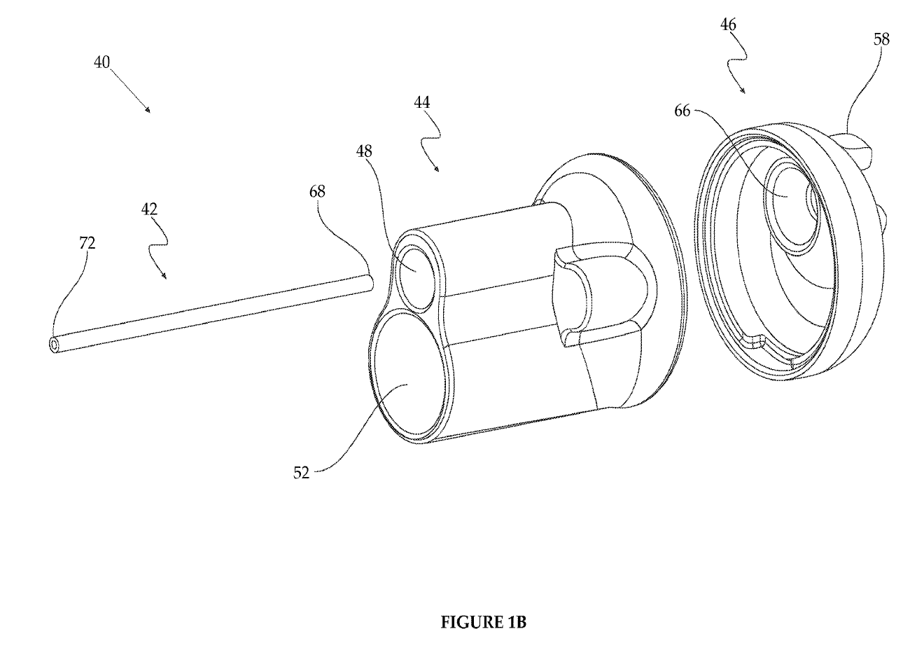

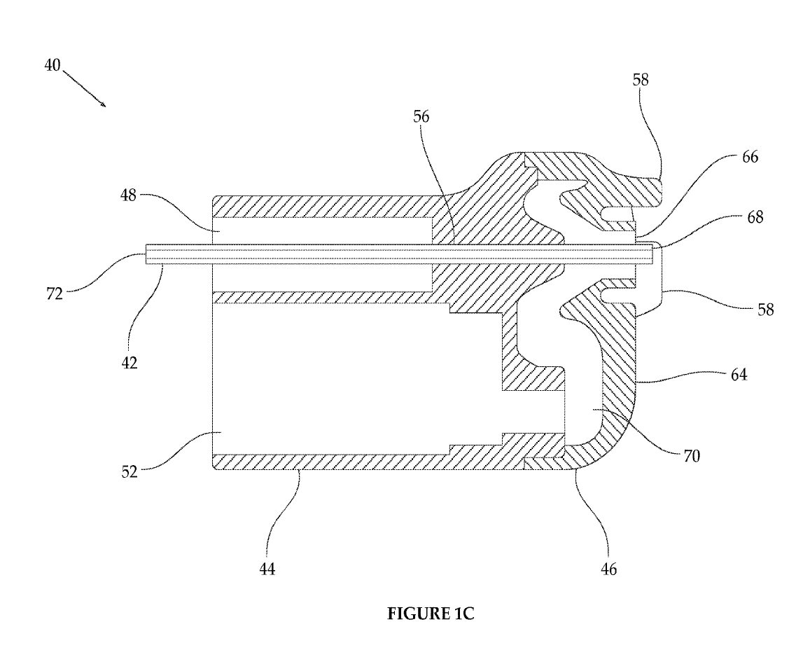

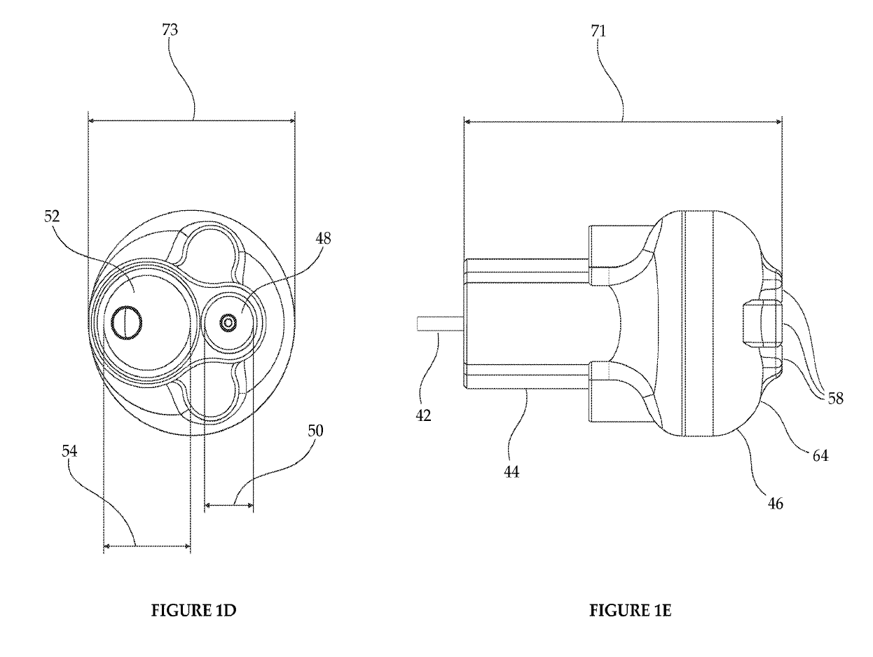

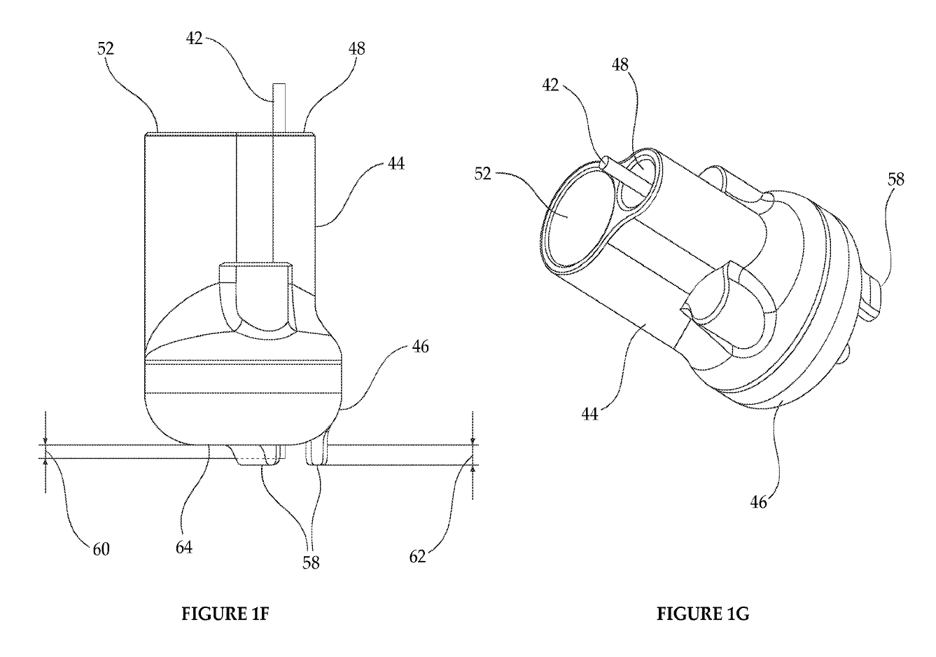

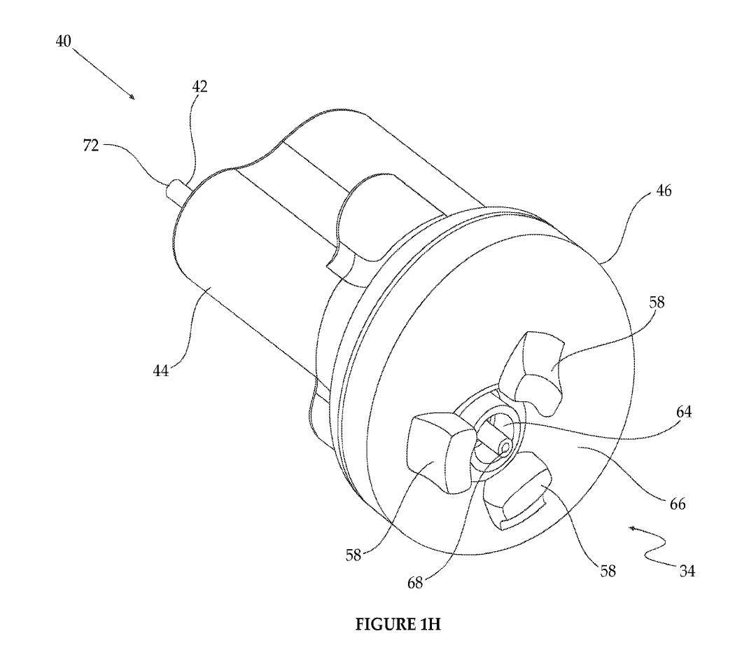

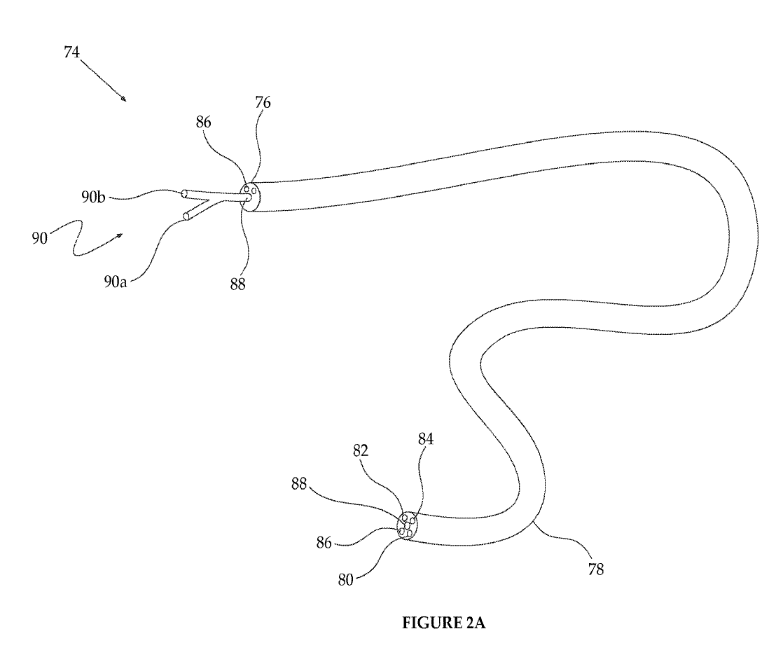

An embodiment of a device 10 useful in endoscopy as described herein is described with reference to FIGS. 1A-1I. Device 10 is configured for use with a standard colonscope.

Device 10 is schematically depicted in FIG. 1A. Device 10 comprises a flexible elongated body 12 including: a 1.4 mm outer-diameter (OD)/1.2 mm inner-diameter (ID) polyethylene gas-conveying tube 14 which bore constitutes a gas channel 16, a 0.8 mm OD/0.5 mm ID polyethylene liquid-conveying tube 18 which bore constitutes a liquid channel 20 both passing through the bore of a 3.3 mm OD/3.0 mm ID polyethylene tube 22 that constitutes a sheath defining some of the outer surface of elongated body 12, so that gas channel 16 and liquid channel 20 are parallel.

At a proximal end 24 of elongated body 12 is an adaptor 26 for functionally associating gas-conveying tube 14 with a gas-source and an adaptor 28 for functionally associating liquid-conveying tube 18 with a liquid-source.

At a distal tip 30 of a distal end 32 of elongated body 12 is a nozzle 34 functionally associated with gas channel 16 and liquid channel 20. Nozzle 34, together with the other components of device 10, is configured to generate a liquid-gas stream comprising a mist from a gas conveyed by gas channel 16 from a pressurized gas-source and a liquid conveyed by liquid channel 20 from a pressurized or a non-pressurized liquid-source, in a direction parallel to the axis of distal end 32. Nozzle 34, together with the other components of device 10, is configured so that gas conveyed to nozzle 34 through gas channel 16 draws a liquid (by a Venturi effect) through liquid channel 20 to nozzle 34 to generate a liquid-gas stream.

Device 10 further includes an activation mechanism comprising a gas cut-off valve 36 for controlling generation of a liquid-gas stream. When the activation mechanism is in an off state, gas cut-off valve 36 is closed, blocking conveying of gas to nozzle 34 thereby preventing generation of a liquid-gas stream. When the activation mechanism is in an on state, gas cut-off valve 36 is open and allows conveying of gas to nozzle 34. Gas cut-off valve 36 is biased to the closed-state by a mechanical spring. An operator depresses a foot pedal 38 to open gas cut-off valve 36 and releases foot pedal 38 to allow gas cut-off valve 36 to move back to the closed-state.

A nozzle assembly 40 that defines nozzle 34 is depicted in greater detail in FIGS. 1B to 1H.

In FIG. 1B, nozzle assembly 40 is depicted exploded to three constituent parts: liquid discharge tube 42 (0.2 mm OD/0.1 mm ID, 6 mm long stainless steel tube), base 44 and cap 46, both of injection-molded polycarbonate.

In FIG. 1C, nozzle assembly 40 is depicted fully-assembled in side cross-section.

Nozzle assembly 40 is depicted assembled in greater detail in FIG. 1D (back view), FIG. 1E (top view), FIG. 1F (side view), FIG. 1G (perspective view from a back quadrant) and FIG. 1H (perspective view from a front quadrant).

Base 44 defines, inter alia, a liquid inlet 48 having a diameter 50 of 0.8 mm to accommodate the distal end of liquid-conveying tube 18, a gas inlet 52 having a diameter 54 of 1.4 mm to accommodate the distal end of gas-conveying tube 14, and a 0.2 mm diameter discharge tube-retaining passage 56.

Cap 46 defines, inter alia, three tube protectors 58 0.3 mm 62 from a distal surface 64 of cap 46, and a gas-discharge aperture 66 having a diameter of 0.5 mm.

For assembly of nozzle assembly 40, base 44 and cap 46 are snapped together so that discharge tube-retaining passage 56 is coaxial with gas-discharge aperture 66. Liquid discharge tube 42 is firmly held in discharge tube-retaining passage 56 (see FIG. 1C) and passes coaxially through the center of gas-discharge aperture 66 to protrude 0.2 mm 60 from distal surface 64 of cap 46.

When nozzle assembly 40 is fully assembled, a distal tip 68 of liquid discharge tube 42 constitutes a liquid outlet, and base 44 together with cap 46 define chamber 70 (see FIG. 1C) in fluid communication with gas inlet 52 (constituting the inlet of chamber 70) and in fluid communication with the outside through gas-discharge aperture 66. When fully assembled, nozzle assembly has a length 71 of 5.10 mm and a diameter 73 of 3.3 mm.

When device 10 is fully assembled for use, the distal end of gas-conveying tube 14 is placed in and securely held in gas inlet 52 and the distal end of liquid-conveying tube 16 is placed in and securely held in liquid inlet 48.

For use of device 10, a suitable gas is conveyed from a pressurized gas-source (e.g., a bottle of carbon dioxide with a regulator) through gas channel 16 of gas-conveying tube 14. The gas leaves gas channel 16 to enter chamber 70 and exits chamber 70 through gas-discharge aperture 66 to ambient. Preferably, the pressure of the gas in chamber 70 is sufficiently higher than ambient so that the flow of gas through gas-discharge aperture 66 is choked flow so that the gas velocity through the narrowest portion of gas-discharge aperture 66 is sonic or near-sonic (approaches local speed of sound). As the gas expands out of the narrowest portion of gas-discharge aperture 66, the gas flow increases to supersonic velocity while the pressure around distal tip 68 of liquid discharge tube 42 drops due to the Venturi effect.

Simultaneously, a suitable liquid is conveyed from a liquid-source (e.g., a pressurized liquid-source such as a syringe pump or a bag of saline maintained higher (e.g., a meter) than nozzle 34, or a non-pressurized liquid-source such as a bag of saline maintained at the same height or lower than nozzle 34) through liquid channel 20 of tube 18 to liquid inlet 48. The liquid passes through liquid discharge tube 42 from a proximal tip 72 to distal tip 68 by a combination of capillary action and the low pressure around distal tip 68.

The gas flow from gas-discharge aperture 66 around distal end 68 draws liquid from distal tip 68 as small particles (typically having a mean particle diameter of less than about 20 micrometers), thereby generating a desired liquid-gas stream comprising a mist of particles of liquid in the gas. Due the conditions of the gas flow, the generated gas-liquid stream is of constant intensity (e.g., no sputtering) in the shape of a cone having a divergence of 11° from parallel along the axis of nozzle assembly 40 and distal end 32 of elongated body 12.

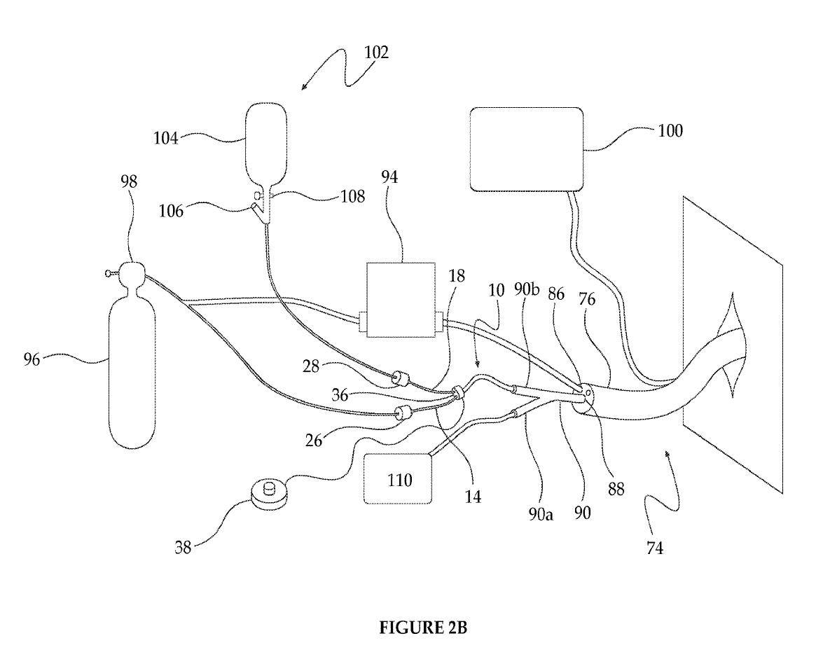

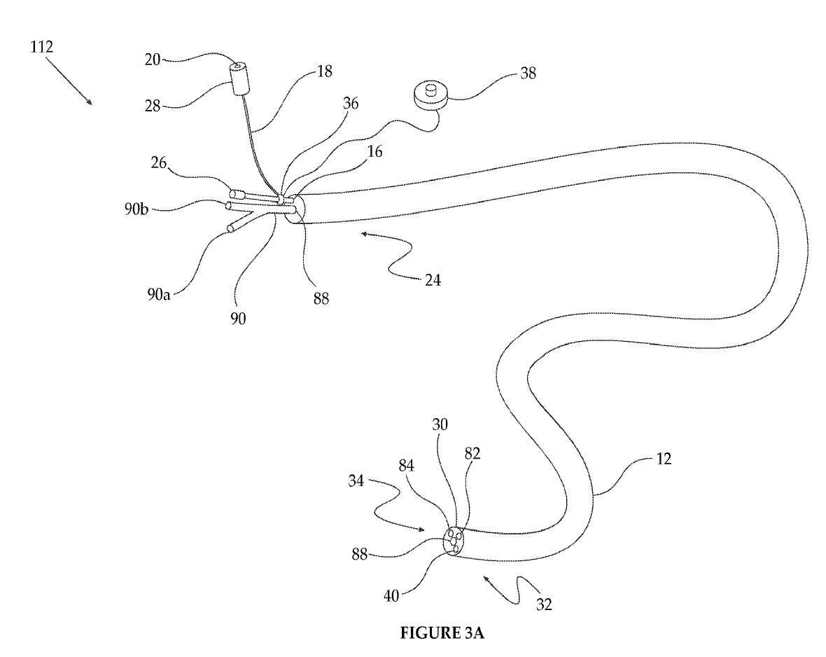

With reference to FIG. 2, the use of device 10 with a colonoscope 74 (a Pentax HD+-colonoscopes (Hi-line series by Pentax, Tokyo, Japan) having a standard 3.7 mm working channel) with a proximal end 76 and a distal end 78 is discussed. In FIG. 2A, colonoscope 74 is schematically depicted. In FIG. 2B, device 10 is schematically depicted together with colonoscope 74.

As known in the art, at a distal tip 80 of colonoscope 74 is an illuminator 82 and an imager 84 (a video camera). Additionally, there are three channels passing from proximal end 76 to distal end 78, opening out at distal tip 80: an insufflation channel 86 for conveying an insufflation gas from an insufflation gas-source to distal tip 80, a washing channel (not numbered) for conveying a pressurized stream of liquid from a washing liquid-source to distal tip 80 and a 3.8 mm diameter working channel 88 accessible through an adaptor 90 allowing application of suction through port 90a or introduction an auxiliary tool such as device 10 through port 90b.

A patient is prepared in the usual way for a colonoscopy and colonoscope 74 is maneuvered in the usual way into the gastrointestinal tract. An insufflation gas-source 94 (a known insufflation controller functionally associated with a bottle of compressed carbon dioxide 96 provided with a pressure regulator 98) is activated so that insufflation gas (carbon dioxide) is conveyed into and insufflates the gastrointestinal tract. The illuminator and imager are activated allowing acquisition of images as streaming video for display on a display screen 100.

Device 10 is prepared for use. Bottle 96 of compressed carbon dioxide and pressure regulator 98, constituting a pressurized gas-source, are functionally associated with gas channel 16 through adaptor 26 and a pressurized liquid-source 102 (a collapsible bag of saline 104 with an injection port 106 and a valve 108 suspended about 1 meter higher than the patient) is functionally associated with liquid channel 20 through adaptor 28.

Regulator 98 is opened to provide a desired gas pressure in gas channel 16. Valve 108 of liquid-source 102 is opened. Foot pedal 38 is depressed to open gas cut-off valve 36 to an open state. When valve 36 is open, gas from bottle 96 is conveyed through gas channel 16, and out through nozzle 34, generating lower pressure that draws liquid from liquid-source 102 through liquid channel 20 to nozzle 34. When it is observed that opening of gas cut-off valve 36 leads to generation of a liquid-gas stream directed from nozzle 34, foot pedal 38 is released, so gas cut-off valve 36 returns to the normally closed state.

Distal tip 30 first, distal end 32 of device 10 is introduced into working channel 88 of colonoscope 74 through port 90b of adaptor 90 and advanced until the distal tip of nozzle assembly 40 just emerges from distal tip 80 of colonoscope 74 with reference to markings (not depicted) on the outer surface of elongated body 12 of device 10.

The operator performs a colonoscopy in the usual way with reference to the images acquired by illuminator 82 and displayed on display screen 100, maneuvering distal end 78 of colonoscope 74 and therefore distal end 32 of device 10 in the gastrointestinal tract. The operator optionally uses device 10 to implement aspects of the method as described herein.

For example, if an object such as feces is observed obstructing the view of a portion of the surface of the gastrointestinal wall (for example is lodged in a crack or crevice), the operator may choose to direct distal end 78 of colonoscope 74 to proximity of the object and depresses foot pedal 38, opening gas cut-off valve 36 to generate a liquid-gas stream that moves (in some cases also disintegrates) the object.

If the force applied by the liquid-gas stream is insufficient to dislodge the object and/or the operator wants to retain a wide view through illuminator 82, the operator optionally advances device 10 through working channel 88 under guidance of the images displayed on display screen 100 so that nozzle 34 is closer to the object and then depresses foot pedal 38 to generate a liquid-gas stream.

If desired, the operator can slightly withdraw device 10 so that nozzle 34 is retracted inside working channel 88 and activate a suction device 110 to apply suction through working channel 88 to remove objects and fragments of objects from the gastrointestinal tract in the usual way. If the suction force is insufficient, the operator can optionally withdraw device 10 entirely out of working channel 88, perform suction in the usual way, and then reintroduce device 10 into working channel 88 through port 90b of adaptor 90.

If the operator observes a bleeding lesion which bleeding obstructs visual inspection, the operator may choose to direct distal end 78 of colonoscope 74 to proximity of the bleeding lesion and depresses foot pedal 38 to generate a liquid-gas stream that moves the blood away and cools the bleeding blood vessels. The blood vessels contract so the bleeding stops, allowing visual inspection of the lesion and surroundings.

If the operator observes excisable tissue (e.g., a polyp), the operator may choose to withdraw device 10 entirely out of working channel 88, and introduce an excising tool (not depicted) into working channel 88 through port 90b of adaptor 90 in the usual way. The operator uses the excising tool in the usual way to excise the excisable tissue and then withdraws the excising tool. The operator reintroduces device 10 into working channel 88 through port 90b of adaptor 90. If bleeding from the area from which the tissue was excised is observed, the operator may choose to direct distal end 78 of colonoscope 74 to proximity of the bleeding and depresses foot pedal 38 to generate a liquid-gas stream that cools the bleeding blood vessels. The blood vessels contract so the bleeding stops.

If the operator observes a portion of the surface of the gastrointestinal wall which is desired to mark with a gastrointestinal stain, the operator may choose to introduce a liquid including the desired stain to liquid channel 20 through injection port 106 of liquid-source 102, directs distal end 78 of colonoscope 74 to proximity of the portion of the surface of the gastrointestinal wall and depresses foot pedal 38 to generate a liquid-gas stream that applies the stain to the portion of the surface of the gastrointestinal wall. Alternatively, the operator may choose to disconnect liquid-source 102 from adaptor 28, functionally associate an alternative liquid-source (not depicted) that includes a desired stain with adaptor 28, generate a liquid-gas stream with the liquid from the alternative liquid-source to apply the stain as described above, detach the alternative liquid-source and then reassociate liquid-source 102 with adaptor 28.

If the operator observes a portion of the surface of the gastrointestinal wall to which it is desired to administer an active pharmaceutical ingredient, the operator may choose to introduce a pharmaceutical composition including the active pharmaceutical ingredient to liquid channel 20 through injection port 106 of liquid-source 102, directs distal end 78 of colonoscope 74 to proximity of the portion of the surfaces of the gastrointestinal tract and depresses foot pedal 38 to generate a liquid-gas stream that administers the active pharmaceutical ingredient to the portion of the surface of the gastrointestinal wall. Alternatively, the operator may choose to disconnect liquid-source 102 from adaptor 28, functionally associate an alternative liquid-source (not depicted) that includes a desired active pharmaceutical ingredient with adaptor 28, generate a liquid-gas stream with the liquid from the alternative liquid-source to administer the active pharmaceutical ingredient as described above, detach the alternative liquid-source and then reassociate liquid-source 102 with adaptor 28.

Gas-cut off valve 36 of device 10 discussed above is configured to prevent conveying of gas through gas channel 16 when in a closed state, thereby preventing generation of a liquid-gas stream. In some related embodiments, gas-cut off valve 36 is additionally configured to function as a liquid cut-off valve to concurrently prevent conveying of liquid through liquid channel 20 when in a closed state.

In some related embodiments, a device includes, in addition to gas-cut off valve 36, a separate liquid cut-off valve configured to concurrently prevent conveying of liquid through liquid channel 20 when in a closed state. In some such embodiments, the device is configured to allow the gas-cut off valve and the liquid cut-off valve to be opened and closed independently while in some such embodiments, the device is configured to concurrently open or close both the gas-cut off valve and the liquid cut-off valve.

Device 10 discussed above comprises an activation mechanism and is thereby configured for generation of a liquid-gas stream on-demand. Specifically, gas cut-off valve 36 prevents generation of a liquid-gas stream by preventing conveying of gas through gas channel 16. In some embodiments, a device such as 10 is operated so that a liquid-gas stream is generated continuously, for example, by removing gas cut-off valve 36, locking gas cut-off valve 36 in an open state, or continuously depressing foot pedal 38. Some embodiments of a device as described herein lack an activation mechanism and are thereby configured for operation with continuous generation of a liquid-gas stream. Any advantage of some such embodiments is simplicity and the fact that the gas component of the liquid-gas stream released inside the bodily cavity can substantially supplement or even entirely replace dedicated insufflation, for example, the conveying of insufflation gas by insufflation gas-source 94 through insufflation channel 86 of colonoscope 74. In some such embodiments, the gas channel such as 16 constitutes an insufflation channel.

Device 10 discussed above is configured for use with an endoscope includes only two channels: gas channel 16 and liquid channel 20. When device 10 is used, insufflation is performed, for example, using the insufflation channel of the endoscope such as 74 with which used, or insufflation gas is conveyed through gas channel 16 at a rate (e.g., by continuous generation of a liquid-gas stream) so that gas channel 16 constitutes an insufflation channel. In some not-depicted embodiments, a device such as 10 has a separate insufflation channel, implemented (for example) with an additional gas-conveying tube passing through sheath 22 defining a dedicated insufflation channel with a separate insufflation gas outlet at distal end 32 of elongated body 12, for example, an insufflation gas outlet formed through nozzle assembly 40 or formed through the distal end of sheath 22.

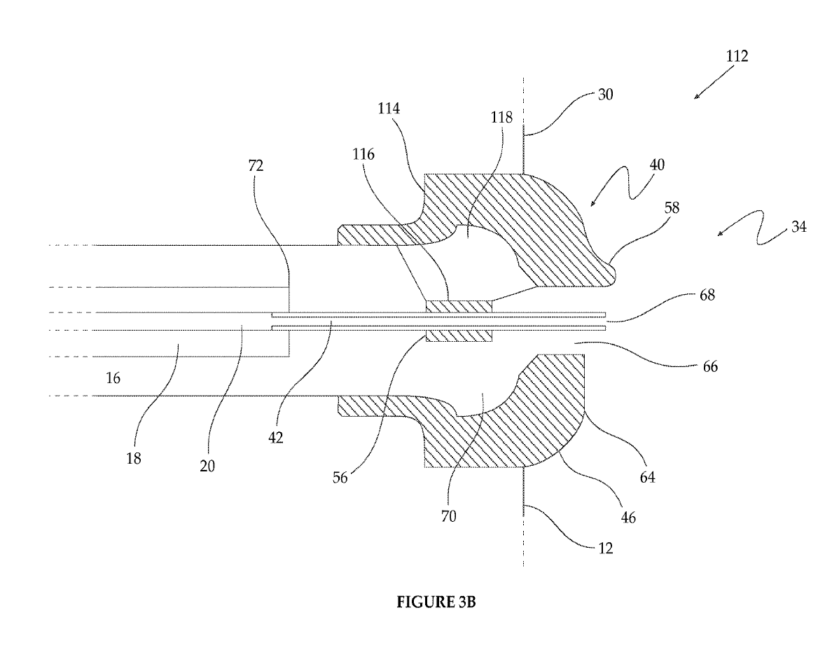

An additional embodiment of a device as described herein, a device 112, is schematically depicted in FIG. 3A. In FIG. 3B, a portion of a distal end 32 of device 112 is schematically depicted in side cross section to show details of nozzle 34.

Device 112 substantially constitutes a gastrointestinal endoscope (specifically, a flexible gastroscope) and is similar in construction and design to known gastroscopes such as a Pentax EG-2901. Device 112 comprises a flexible elongated body 12 1.05 m long and having a 9.8 mm outer diameter. At a distal tip 30 of distal end 32 of elongated body 12 of device 112 is an illuminator 82 (a light emitting diode), an imager 84 (a camera).

Passing from a proximal end 24 to a distal end 32 of elongated body 12 opening out at distal tip 30 are three channels:
a gas channel 16, a 1.7 mm diameter channel passing through elongated body 12;
a liquid channel 20 defined by the bore of a 0.8 mm OD/0.5 mm ID polyethylene liquid-conveying tube 18, disposed inside gas channel 16 so that gas channel 16 and liquid channel 20 are substantially coaxial; and
a 2.5 mm diameter working channel 88 passing through elongated body 12 accessible through an adaptor 90 allowing application of suction through port 90a or introduction an auxiliary tool through port 90b.

Device 112 further comprises a gas cut-off valve 36 for controlling generation of a liquid-gas stream. When closed, liquid cut-off valve 36 prevents generation of a liquid-gas stream by preventing conveying of liquid to nozzle 34 by compressing liquid channel 20 while when open liquid cut-off valve 36 allows conveying of liquid to nozzle 34 and thereby generation of a liquid-gas stream. Liquid cut-off valve 36 comprises a solenoid biased to the closed-state by a mechanical spring. An operator uses foot pedal 38 open liquid cut-off valve 36 and releases foo-pedal 38 to allow activation valve 36 to close. It is important to note that the conveying of gas through gas channel 16 is uninterrupted by liquid cut-off valve 36.

As seen in FIG. 3B, at distal end 32 of elongated body 12, gas channel 16 opens up to define a wider cavity 114. Pressed into and thereby retained in cavity 114 is nozzle assembly cap 46 that together with a liquid discharge tube 42 (identical to tube 42 of device 10) constitute a nozzle assembly 40 that substantially defines nozzle 34 of device 112.

Nozzle assembly cap 46 defines, inter alia, three tube protectors 58 protruding 0.3 mm from a distal surface 64 of cap 46, a gas-discharge aperture 66 having a diameter of 0.5 mm, a chamber 70 and a tube retaining passage 56 coaxial with gas-discharge aperture 66 defined by tubular support 116 and held in place by three flanges 118 (one visible in FIG. 3B).

When nozzle assembly 40 is fully assembled, a proximal tip 72 of liquid discharge tube 42 is in functionally associated with a distal end of liquid conveying tube 18 so that the bore of liquid discharge tube 42 is in fluid communication with liquid channel 20, allowing passage of liquid conveyed through liquid channel to a distal tip 68 of liquid discharge tube 42 that constitutes a liquid discharge aperture. Liquid discharge tube 42 passes through and is held in place by tube retaining passage 56 to be coaxial with gas-discharge aperture 66 and protruding 0.2 mm from a distal surface 64 of cap 46.

The operation of nozzle 34 defined by nozzle assembly 40 of device 112 is substantially the same as of device 10.

The use of device 112 is substantially analogous to the use of device 10 with a colonoscope such as 74. That said, one substantial difference is, as noted above, that liquid cut-off valve 36 does not interrupt the conveying of gas through gas channel 16. Accordingly, during use of device 112, a continuous stream of gas is released from nozzle 34, the gas effective for insufflation of the bodily cavity in which located. Opening of liquid cut-off valve 36 allows liquid to be conveyed through liquid channel 20 to nozzle 34 to generate a liquid-gas stream when needed, for example, to implement an embodiment of a method as described herein.

Unlike known endoscopes, device 112 does not include a dedicated insufflation channel, rather gas channel 16 constitutes an insufflation channel. In related embodiments, a device 112 includes a dedicated insufflation channel as known in the art of endoscopes, for example, such as insufflation channel 86 of endoscope 74 in FIG. 2A. Such a dedicated insufflation channel can be used for insufflation of a bodily cavity instead of or in addition to gas channel 16.

Both device 10 and device 112 are configured for use with both a pressurized and a non-pressurized liquid source. When used with a non-pressurized liquid-source, operation of the devices relies on the Venturi effect to draw liquid from a liquid channel 20 to generate a liquid-gas stream. In some embodiments, a device such as 10 or 112 is used with a pressurized liquid-source to apply pressure to liquid in the liquid channel 20 to force the liquid therethrough, thereby conveying the liquid to a nozzle 34. In some such embodiments, the activation mechanism of the device further comprises a liquid cut-off valve. In some such embodiments where the liquid-source is a pump, the activation mechanism functions by deactivating the pump (so that no liquid is conveyed to the nozzle and consequently no liquid-gas stream generated) or activating the pump (so that liquid is conveyed to the nozzle and consequently a liquid-gas stream generated).

In the embodiments of a device as discussed above, a device comprises two channels, a gas channel and a liquid channel. In some embodiments, a device comprises a single channel which distal end is functionally associated with a nozzle, the single channel configured for conveying a liquid-gas stream from a proximal end of the channel to the nozzle, so that a liquid-gas stream emerging from the distal end of the device is directed by the nozzle.

### EXPERIMENTAL

48 patients scheduled to undergo screening or surveillance (after previous polypectomy) endoscopy were instructed to perform pre-colonoscopy bowel cleansing by their primary care physicians or according to the routine hospital protocol using three liters of Polyethylene glycol giving the evening before the procedure.

Colonoscopy under conscious sedation using Propofol was performed using Pentax HD+-colonoscopes (Hi-line series by Pentax, Tokyo, Japan). The colonoscope was inserted via the anus and advanced to the cecum or terminal ileum. The degree of the colon cleansing was assessed during using the Ottawa scale. Patients having diverticulosis (6), visible polyps greater than 0.5 cm (4) or who showed sufficient colon cleanliness (6 Ottawa scale ≦4) were excluded.

The remaining 32 patients (14 female, mean age 61 years, 22 screening, 10 surveillance) were examined in accordance with the teachings herein. The average colon cleanliness of the 32 patients was 9.4 on the Ottawa scale.

A device such as described with reference to FIG. 1 was passed through the working channel of the colonoscope to the distal end of the colonoscope. As the colonoscope was withdrawn, the device was used to generate a liquid-gas stream at any obscuring objects seen, typically feces lodged in folds and crevices of the colon. It was observed that the feces was disintegrated and effectively removed from folds and crevices of the colon.

In some instances, fecal fragments as well as residual liquids were removed from the colon by suction in the usual way by suction through the working channel while the device was located in the working channel. In some instances, the device was removed from the working channel during suction to increase suction power.

After implementation of the teachings herein, the average colon cleanliness of the 32 patients achieved using the teachings herein was a statistically significant 6.8 on the Boston scale, allowing increased visibility of the colon mucosal surface.

Due to implementation of the teachings herein, withdrawal of the endoscope was prolonged (by 11.4±6.0 minutes). That said, all patients tolerated the procedure well and no device-related adverse or serious adverse events were noted.

It was observed that the cleaning using the teachings herein was highly effective, providing significant improvement of colon cleaning. Importantly, 18 adenomas (2 advanced) and 1 colon cancer in 11 patients that were otherwise obscured and undetectable were only identified as a result of the use of the teachings herein.

A person having ordinary skill in the art is able to implement the teachings herein, especially with reference to the art of endoscopes and U.S. Pat. No. 6,283,936; U.S. Pat. No. 6,673,081; PCT Patent Application PCT/IL2005/000017; PCT Patent Application PCT/IL2006/000557 and US patent publication 2009/0234269 (all which are included by reference as if fully set forth herein). Devices as described herein may be fashioned of any suitable material. For example, in embodiments in embodiments including a liquid discharge tube, such as described in the embodiments depicted above as fashioned from stainless steel, a component equivalent to a liquid discharge tube may be fashioned of any suitable material, for example, any suitable a metal or polymer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

In case of conflict with documents included by reference, the specification, including definitions, takes precedence.

1. A device useful in endoscopy, comprising:
a) an elongated body having a proximal end and a distal end configured for maneuvering said distal end into a bodily cavity;
b) a nozzle at said distal end of said elongated body;
c) a gas channel configured for conveying a gas from said proximal end of said elongated body to said nozzle; and
d) a liquid channel configured for conveying a liquid from said proximal end of said elongated body to said nozzle simultaneously with conveying of said gas;
wherein said nozzle is configured to generate a liquid-gas stream comprising a mist of particles of liquid in said gas directed in a specified direction from a gas conveyed by said gas channel and a liquid conveyed by said liquid channel, the directed liquid-gas stream sufficient for moving a resting object from a surface of a bodily cavity.

2. The device of claim 1, substantially constituting an endoscope.

3. The device of claim 1, wherein said elongated body is configured for passage through a channel of an endoscope, distal end first, from a proximal end to a distal end thereof.

4. The device of claim 1, wherein said gas channel and said liquid channel are substantially coaxial.

5. The device of claim 1, wherein said gas channel and said liquid channel are substantially parallel.

6. The device of claim 1, configured so that a said mist comprises particles of liquid having a mean particle diameter of less than about 20 micrometers.

7. The device of claim 1, configured to generate a liquid-gas stream having a velocity relative to said nozzle in said specified direction in the range of from about about 100 m/sec to about 300 m/sec.

8. The device of claim 1, further comprising a gas-source functionally associated with the gas channel at the proximal end of the distal body to provide a gas to be conveyed by said gas channel.

9. The device of claim 8, wherein said gas comprises medical-grade carbon dioxide.

10. The device of claim 1, configured so that a flow rate of a said gas in a said generated liquid-gas stream from said nozzle is in the range of from about 0.5 l/minute to about 2 l/minute.

11. The device of claim 1, configured so that a said gas is conveyed to said nozzle at a pressure in the range of from about 5 kPa to about 14 kPa above ambient.

12. The device of claim 1, configured so that a flow rate of a said liquid in a said generated liquid-gas stream from said nozzle is in the range of from about 0.1 ml/minute to about 2 ml/minute.

13. The device of claim 1, configured for use with a non-pressurized liquid-source for providing a said liquid to be conveyed by said liquid channel.

14. The device of claim 13, configured so that a said gas conveyed by said gas channel to said nozzle draws a said liquid through said liquid channel to said nozzle to generate a said liquid-gas stream.

15. The device of claim 1, further comprising an activation mechanism for controlling generation of a said liquid-gas stream having at least two states, an off state during which a said liquid-gas stream is not generated and an on state during which a said liquid-gas stream is generated, wherein in said off state, said activation mechanism prevents said generation of said liquid-gas stream by preventing said conveying of a said gas to said nozzle.

16. The device of claim 1, further comprising an activation mechanism for controlling generation of a said liquid-gas stream having at least two states, an off state during which a said liquid-gas stream is not generated and an on state during which a said liquid-gas stream is generated, wherein in said off state, said activation mechanism prevents said generation of said liquid-gas stream by preventing said conveying of a said liquid to said nozzle.

17. The device of claim 16, configured so that conveying of a said gas to said nozzle is optionally uninterrupted when said activation mechanism is in said off state.

18. The device of claim 1, further comprising an insufflation channel for conveying an insufflation gas from said proximal end to said distal end of said elongated body.

19. The device of claim 18, wherein said gas channel constitutes said insufflation channel.

20. A method for performing endoscopy, comprising:
a) directing a distal end of an endoscope to proximity of a portion of a surface of a bodily cavity; and
b) directing a liquid-gas stream comprising a mist of particles of liquid in a gas at said portion of said surface of said bodily cavity.
